(19) United States
(12) Patent Application Publication
(10) Pub. No.: US 2008/0003625 A1
(43) Pub. Date: Jan. 3, 2008

(54) Predicting Alzheimer's Disease

(76) Inventors: Steven G. Younkin, Ponte Vedra, FL (US); Linda H. Younkin, Ponte Vedra, FL (US); Neill Graff-Radford, Jacksonville, FL (US); Julia E. Crook, Jacksonville, FL (US); John A. Lucas, Jacksonville, FL (US)

Correspondence Address: FISH & RICHARDSON P.C., PO BOX 1022, MINNEAPOLIS, MN, 55440-1022, US

(21) Appl. No.: 11/572,253

(22) PCT Filed: Jul. 19, 2005
(86) PCT No.: PCT/US05/25723

§ 371 Date: Sep. 24, 2007

Related U.S. Application Data
(60) Provisional application No. 60/589,410, filed on Jul. 19, 2004.

(51) Int. Cl. G01N 33/53

(52) U.S. Cl. 435/7.92; 436/86

(57) ABSTRACT

This document relates to methods and materials related to determining whether or not a mammal (e.g., a human) is susceptible to develop mild cognitive impairment or Alzheimer's disease.

### BACKGROUND

1. Technical Field

This document relates to methods and materials involved in determining whether or not a human is susceptible to develop mild cognitive impairment or Alzheimer's disease.

2. Background Information

Alzheimer's disease (AD) is the most common form of age-related neurodegenerative illness. The defining pathological hallmarks of AD are the presence of neurofibrillary tangles and senile plaques in the brain. Amyloid β polypeptides (Aβ) are the major constituents of amyloid plaques and are derived from altered processing of amyloid precursor proteins (APPs). Aβ consists predominantly of two forms, Aβ₄₀ and Aβ₄₂. Although Aβ₄₀ is the predominant form, recent evidence suggests that Aβ₄₂ is the pathogenic form.

### SUMMARY

This document involves methods and materials related to determining whether or not a mammal (e.g., a human) is susceptible to develop mild cognitive impairment (MCI) or Alzheimer's disease (AD). Identifying at-risk individuals can allow doctors and patients to select treatment and patient care options before the onset of noticeable or impairing symptoms. Analyses of plasma Aβ have shown that Aβ₄₂ is increased in subjects who have PS1/2 or APP717 mutations that cause early onset familial AD. Both Aβ₄₂ and Aβ₄₀ are elevated in plasma from subjects with Down's syndrome or the Swedish APP mutation, and both forms are also elevated in the unaffected first-degree relatives and extended families of patients with late onset AD. In addition, plasma Aβ₄₀ increases with aging over age 65. Aβ₄₂ may also increase, but to a lesser extent.

Plasma Aβ₄₂ is not significantly increased in patients with AD. Many studies, however, have shown that CSF Aβ₄₂ is significantly decreased in AD patients and MCI patients. In the Tg2576 transgenic mouse model of AD, both CSF Aβ and plasma Aβ levels were found to decrease as Aβ is deposited in the brain.

As described herein, over five hundred normal Caucasian subjects were followed for up to 12 years. The subjects were cognitively tested about every one to two years. From this study, the ratio of plasma Aβ₄₂/Aβ₄₀ was found to be a strong predictor of conversion to AD or MCI.

In general, this document features a method for determining whether or not a human is susceptible to develop mild cognitive impairment or Alzheimer's disease. The method includes obtaining a plasma sample from the human and determining the Aβ₄₂ to Aβ₄₀ ratio in the plasma sample, wherein a ratio less than 0.082 indicates that the human is susceptible to develop mild cognitive impairment or Alzheimer's disease. An Aβ₄₂ to Aβ₄₀ ratio less than 0.05 can indicate that the human is susceptible to develop mild cognitive impairment or Alzheimer's disease within two years.

In another embodiment, this document features a method for determining whether or not a human is susceptible to develop mild cognitive impairment or Alzheimer's disease. The method includes (a) determining the Aβ₄₂ to Aβ₄₀ ratio in a plasma sample from the human, and (b) classifying the human as being or not being susceptible to develop mild cognitive impairment or Alzheimer's disease, wherein the human is susceptible to develop mild cognitive impairment or Alzheimer's disease if the Aβ₄₂ to Aβ₄₀ ratio in the plasma sample is less than 0.082, and wherein the human is not susceptible to develop mild cognitive impairment or Alzheimer's disease if the Aβ₄₂ to Aβ₄₀ ratio in the plasma sample is greater than 0.082. The human can be classified as being susceptible to develop mild cognitive impairment or Alzheimer's disease within two years if the Aβ₄₂ to Aβ₄₀ ratio is less than 0.05. The human can be classified as being susceptible to develop mild cognitive impairment or Alzheimer's disease within four years if the Aβ₄₂ to Aβ₄₀ ratio is between 0.05 and 0.064. The human can be classified as being susceptible to develop mild cognitive impairment or Alzheimer's disease within ten years if the Aβ₄₂ to Aβ₄₀ ratio is between 0.064 and 0.082.

In another embodiment, this document features a method for determining whether or not a mammal is susceptible to develop mild cognitive impairment or Alzheimer's disease. The method includes determining whether or not the mammal contains plasma with an Aβ₄₂ to Aβ₄₀ ratio that is less than 0.082, wherein the presence of the ratio indicates that the mammal is susceptible to develop mild cognitive impairment or Alzheimer's disease. The mammal can be a human. The mammal can be a human not diagnosed with mild cognitive impairment or Alzheimer's disease. The method can include determining whether or not the mammal is susceptible to develop mild cognitive impairment. The method can include determining whether or not the mammal is susceptible to develop Alzheimer's disease. The method can include determining whether or not the mammal contains plasma with an Aβ₄₂ to Aβ₄₀ ratio that is less than 0.064. The method can include determining whether or not the mammal contains plasma with an Aβ₄₂ to Aβ₄₀ ratio that is less than 0.05. The determining step can include using an enzyme-linked immunosorbant assay. The method can include classifying the mammal as being susceptible to develop mild cognitive impairment or Alzheimer's disease within two years. The method can include classifying the mammal as being susceptible to develop mild cognitive impairment or Alzheimer's disease within two years when the Aβ₄₂ to Aβ₄₀ ratio is less than 0.05. The method can include classifying the mammal as being susceptible to develop mild cognitive impairment or Alzheimer's disease within four years. The method can include classifying the mammal as being susceptible to develop mild cognitive impairment or Alzheimer's disease within four years when the Aβ₄₂ to Aβ₄₀ ratio is between 0.05 and 0.064. The method can include classifying the mammal as being susceptible to develop mild cognitive impairment or Alzheimer's disease within ten years. The method can include classifying the mammal as being susceptible to develop mild cognitive impairment or Alzheimer's disease within ten years when the Aβ₄₂ to Aβ₄₀ ratio is between 0.064 and 0.082. The method can include classifying the mammal as not being susceptible to develop mild cognitive impairment or Alzheimer's disease within ten years when the Aβ₄₂ to Aβ₄₀ ratio is greater than 0.082. The method can include, before the determining step, obtaining a plasma sample from the mammal, wherein the determining step contains determining whether or not the plasma sample contains the Aβ₄₂ to Aβ₄₀ ratio that is less than 0.082.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a graph plotting the estimated cumulative probability of MCI or AD versus time for all subjects.

FIG. 2 is a graph plotting the percent conversion to MCI or AD versus time for subjects that are ApoE4 positive or ApoE4 negative.

FIG. 3 is a graph plotting the estimated cumulative probability of MCI or AD versus time for subjects that have an Aβ₄₂/Aβ₄₀ ratio in the first quartile (Q1), second quartile (Q2), third quartile (Q3), or fourth quartile (Q4).

FIG. 4 is a graph plotting the estimated cumulative probability of MCI or AD versus time for subjects that have the indicated Aβ₄₂/Aβ₄₀ ratio and are either ApoE4 positive or ApoE4 negative.

FIG. 5 is a graph plotting the estimated cumulative probability of MCI or AD versus time for subjects that have the indicated age and Aβ₄₂/Aβ₄₀ ratio.

### DETAILED DESCRIPTION

This document provides methods and materials related to determining whether or not a mammal (e.g., human) is susceptible to develop MCI or AD. Any human can be assessed to determine whether or not he or she is susceptible to develop MCI or AD. For example, humans older than 30, 40, 50, 60, 70, or 80 years old can be assessed to determine their susceptibility to develop MCI or AD. In some cases, the mammal being assessed can be a mammal having no previous symptoms of MCI or AD. For example, normal, healthy humans (e.g., humans with no previous diagnosis of MCI or AD) can be assessed for their susceptibility to MCI or AD. A person's susceptibility to develop MCI or AD can be assessed by measuring plasma levels of Aβ₄₀ and Aβ₄₂ so that the ratio of Aβ₄₂ to Aβ₄₀ can be determined. Any method can be used to measure Aβ₄₀ and Aβ₄₂ levels. For example, ELISAs and other immunological assays can be used to measure Aβ₄₀ and Aβ₄₂ levels within a sample. Once the Aβ₄₀ and Aβ₄₂ levels are determined, the ratio of Aβ₄₂ to Aβ₄₀ can be determined. As described herein, humans with a low Aβ₄₂/Aβ₄₀ ratio can be more susceptible to develop MCI or AD than humans with a high Aβ₄₂/Aβ₄₀ ratio. For example, humans with an Aβ₄₂/Aβ₄₀ ratio less than 0.05 can be classified as being susceptible to develop MCI or AD within two years, humans with an Aβ₄₂/Aβ₄₀ ratio between 0.05 and 0.064 can be classified as being susceptible to develop MCI or AD within four years, humans with an Aβ₄₂/Aβ₄₀ ratio between 0.064 and 0.082 can be classified as being susceptible to develop MCI or AD within eight, nine, or ten years, and humans with an Aβ₄₂/Aβ₄₀ ratio greater than 0.082 can be classified as not being susceptible to develop MCI or AD within ten years.

Any population of mammals can be used to identify a particular Aβ₄₂/Aβ₄₀ ratio that corresponds to a particular level of susceptibility. For example, a population of Caucasians, African Americans, Spanish-speaking first generation United States immigrants, or descendents of individuals from the Pacific Rim can be selected and followed as described herein. Briefly, normal, healthy individuals can be identified and monitored over time (e.g., for 5, 10, 15, or more years) for conversion to MCI or AD. In addition, plasma levels of Aβ₄₀ and Aβ₄₂ can be measured and the ratios of Aβ₄₂ to Aβ₄₀ determined at regular intervals (e.g., every year, every other year, etc.). Any method can be used to determine the Aβ₄₂/Aβ₄₀ ratio that correlates with a particular susceptibility to develop MCI or AD. For example, statistical methods can be used to divide a group of subjects into quartiles based on plasma Aβ₄₂/Aβ₄₀ ratios. In some cases, the lowest quartile can have subjects with an Aβ₄₂/Aβ₄₀ ratio less than 0.05, the second quartile can have subjects with an Aβ₄₂/Aβ₄₀ ratio between 0.05 and 0.064, the third quartile can have subjects with an Aβ₄₂/Aβ₄₀ ratio between 0.064 and 0.082, and the fourth quartile can have subjects with an Aβ₄₂/Aβ₄₀ ratio greater than 0.082. Subjects can be classified as being or not being susceptible to develop MCI or AD based on the quartile in which their plasma Aβ₄₂/Aβ₄₀ ratio falls. Any method can be used to associate an Aβ₄₂/Aβ₄₀ ratio with conversion to MCI or AD including, without limitation, those methods provided herein.

In some cases, subjects with an Aβ₄₂/Aβ₄₀ ratio in the lowest quartile can be classified as having a 10% chance of developing MCI or AD in three years. Subjects with an Aβ₄₂/Aβ₄₀ ratio in the second quartile can reach this incidence by approximately five years, and subjects with an Aβ₄₂/Aβ₄₀ ratio in the third or fourth quartile can reach this incidence can reach this incidence in eight years.

In some cases, the plasma Aβ₄₂/Aβ₄₀ ratio can be used as a premorbid biomarker for developing and implementing a preventive approach to AD therapy. For example, the Aβ₄₂/Aβ₄₀ ratio can be used to identify those at risk to develop MCI or AD.

In some cases, a mammal's susceptibility to develop MCI or AD can be determined by assessing the Aβ₄₂/Aβ₄₀ ratio within plasma of the mammal as well as the mammal's ApoE4 genotype. For example, subjects with an ApoE4 allele and an Aβ₄₂/Aβ₄₀ ratio below, e.g., 0.064 can be classified as having a greater likelihood of developing MCI or AD than a subject having either an ApoE4 allele alone or a Aβ₄₂/Aβ₄₀ ratio below 0.064 alone. Such a greater likelihood can be a probability of at least 5, 6, 7, 8, 9, 10, 15, 20, or more percent probability of developing MCI or AD within 2, 3, 4, 5, 6, 7, 8, 9, 10, or more years.

In some cases, a mammal's susceptibility to develop MCI or AD can be determined by assessing the Aβ₄₂/Aβ₄₀ ratio within plasma of the mammal as well as the mammal's age. For example, subjects older than 60, 75, 80, 85, 90, or 95 and having an Aβ₄₂/Aβ₄₀ ratio below, e.g., 0.064 can be classified as having a greater likelihood of developing MCI or AD than a subject being either older than 60, 75, 80, 85, 90, or 95 alone, or having a Aβ₄₂/Aβ₄₀ ratio below 0.064 alone. Such a greater likelihood can be a probability of at least 5, 6, 7, 8, 9, 10, 15, 20, or more percent probability of developing MCI or AD within 2, 3, 4, 5, 6, 7, 8, 9, 10, or more years. In some cases, a mammal's susceptibility to develop MCI or AD can be determined by assessing the Aβ42/Aβ₄₀ ratio within plasma of the mammal, the mammal's age, and the mammal's ApoE4 genotype.

The invention will be further described in the following examples, which do not limit the scope of the invention described in the claims.

### Example 1

### Aβ₄₂/Aβ₄₀ Ratios can Determine Susceptibility to Develop MCI and AD

Elevated plasma Aβ₄₂ levels can be an important risk factor for AD in the early presymptomatic period, and plasma Aβ₄₂ levels can decline in many subjects as Aβ₄₂ is deposited in the brain during the presymptomatic period. To assess this, plasma Aβ₄₀ levels, Aβ₄₂ levels, and Aβ₄₂/Aβ₄₀ ratios were measured in a large longitudinal study.

Five hundred and sixty-three cognitively normal, older adults who entered Mayo Clinic's Alzheimer's Disease Patient Registry (ADPR) as normal controls and had at least two stored plasma specimens were entered into the study. “Cognitively normal adults” are defined as community-dwelling, independently functioning individuals who were examined by their primary physician and met the following selection criteria: (1) no complaints of memory or other cognitive difficulties during the history taking or systems review components of their medical examination, and no findings suggestive of cognitive compromise during the medical examination; (2) no active neurologic or psychiatric conditions with potential to adversely affect cognition; (3) no use of psychoactive medication in an amount sufficient to affect cognition; and (4) normal memory as noted by the informant.

Individuals with prior histories of medical conditions or disorders that may affect cognition (e.g., head injury) were included only if the conditions were no longer active and no evidence of persistent or residual cognitive impairment existed. Individuals with current, chronic medical conditions (e.g., hypertension, diabetes, etc.) were included if their physicians judged their conditions to be well controlled and not affecting cognition.

All subjects underwent baseline neurologic and neuropsychological evaluations. In addition, a study nurse collected collateral information, including family history of dementia, current medications, Clinical Dementia Rating (CDR; Morris, Neurology, 43:2412-4 (1993)), and Neuropsychiatric Inventory (NPI; Cummings et al., Neurology, 44:2308-14 (1994)) from an informant. Activities of daily living were evaluated using Parts A & B of the Record of Independent Living (ROIL; Weintraub, American Journal of Alzheimer's Care and Related Disorders, 7:35-9 (1986)). Data from informants were available for 98% of study subjects. Family members provided information 84% of the time, and close friends provided information 14% of the time. When an informant was not available, information was solicited directly from the subject.

A neurologist reviewed the CDR with the subject and informant and performed a neurologic examination, including the Kokmen Short Test of Mental Status (STMS; Koklmen et al., Arch Neurol, 48:725-8 (1991)), NIH Stroke Scale (Goldstein et al., Archives of Neurology, 46(6):660-2 (1989)), and Unified Parkinson's Disease Rating Scale (UPDRS; Fahn, Elton, and Mot. Unified Parkinson's Disease Rating Scale. In: Fahn S, Marsden C, Calne D, Golstein M, eds. Recent Developments in Parkinson's Disease. New York: McMillan; 1987). An experienced psychometrist then administered a baseline battery of neuropsychological tests, including the Dementia Rating Scale (DRS; Mattis. Dementia Rating Scale. Odessa: Psychological Assessment Resources; 1983), the Auditory Verbal Learning Test (AVLT; Rey, Archives de Psychologie, 28:286-340 (1941)), Weschler Memory Scale-Revised (WMS-R; Wechsler, Wechsler Memory Scale-Revised, New York: The Psychological Corporation; 1987), and Wechsler Adult Intelligence Scale-Revised (WAIS-R; Wechsler, Weschler Adult Intelligence Scale-Revised, New York: The Psychological Corporation; 1981).

Study subjects were contacted annually for re-evaluation. Current medications and family histories of dementia were updated at each follow-up visit. In addition, the CDR, ROIL, NPI, NIH Stroke Scale, UPDRS, STMS, and neuropsychological test battery were repeated.

All baseline and follow-up examinations were reviewed at a consensus conference staffed by neurologists, neuropsychologists, nurses, psychometrists, a geriatrician, and a social worker. Entry criteria were reviewed at baseline, and a CDR of 0 was confirmed for all individuals who were enrolled as normal subjects in the ADPR. To establish the neurologist's diagnosis as the gold standard and avoid problems of diagnostic circularity in later studies of cognitive predictors of differential diagnosis, neuropsychological data were not reviewed and did not contribute to baseline diagnoses.

All diagnostic information obtained at annual follow-up, including neuropsychological data, was reviewed at a consensus conference. Data were compared to baseline studies to evaluate progression to MCI or dementia. Diagnoses of amnestic and non-amnestic MCI were determined using Mayo criteria (Petersen et al., Archives of Neurology, 56:303-8 (1999)). Standard published criteria were used to diagnose possible and probable AD (McKhann et al., Neurology, 34:939-44 (1984)). Subjects who had abnormal clinical findings on follow-up but did not meet established criteria for MCI or dementia were coded as having cognitive impairment of undetermined origin. More than 85% of amnestic MCI cases convert to AD by seven years (Petersen et al., Archives of Neurology, 58(12):1985-92 (2001)). Therefore, incident cases of amnestic MCI and AD were combined as the end point.

For inclusion in the analysis, subjects were required to have been originally recruited as cognitively normal, to have at least 12 months of follow-up after providing the first plasma sample, and to be greater than 55 years of age. The characteristics of the 563 subjects who met these criteria are provided in Table 1. The median follow-up time was 3.7 years (25 to 75% range, 2.6 to 5.9 years). Fifty-three subjects (9%) were diagnosed with amnestic MCI (Petersen et al., Archives of Neurology, 58(12):1985-92 (2001)) or AD (possible or probable) (McKhann et al., Neurology, 34:939-44 (1984)) during their follow-up period. The median age at first plasma sample was 78 years (25 to 75% range, 73 to 83 years). The median age of onset of MCI or AD was 88 years (25 to 75% range, 85 to 92 years).

TABLE 1
Characteristics of the 563 cognitively normal subjects at baseline.

| | |
|---|---|
| Characteristic | |
| Age at first plasma sample | No. (%) |

| | | |
|---|---|---|
| Up to 69 | 23 | (4) |
| 70-74 | 165 | (29) |
| 75-79 | 137 | (24) |
| 80-84 | 145 | (26) |
| 85+ | 93 | (17) |
| Gender (Female) | 349 | (62) |
| ApoE4 | 134 | (24) |

To evaluate the relationship between cognitive change and plasma Aβ levels, a subgroup of cognitively normal individuals was identified whose plasma Aβ levels were measured within six months of the DRS evaluation and who were cognitively normal at the time of these evaluations. Subjects in this group were required to have follow-up DRS evaluations between 3 and 7 years after the baseline visit. If more than one follow-up was available during this time period, then evaluations were selected that were closest to five years from the baseline evaluation. The cohort that met these criteria consisted of 379 persons, 62.5% female, with a mean age of 73.4, and a mean education of 13.4 years.

Blood was drawn from non-fasting subjects using EDTA as anticoagulant. The plasma was then prepared conventionally, divided into 1 mL aliquots, and frozen within 4 hours. One aliquot was used to measure plasma Aβ levels in duplicate using sandwich ELISAs with well-characterized pairs of capture/detection antibodies provided by Takeda Industries, Inc. BAN-50/BA27 is specific for Aβ₄₀, and BNT-77/BC05 is specific for Aβ₄₂ (Scheuner et al., Nature Medicine, 2:864-70 (1996)). Plasma samples from five normal volunteers were also analyzed in duplicate on each plate and used to normalize Aβ₄₀ and Aβ₄₂ values. This normalization procedure reduced plate-to-plate and day-to-day variance and improved inter-subject comparison of the large number of samples analyzed.

The Kaplan-Meier method was used to estimate the distribution of time to development of MCI or AD. Follow-up was considered to begin at the time that the first plasma sample was obtained. Subjects who never developed MCI or AD were censored at the date of last follow-up. Cox proportional hazards models were used to investigate associations with Aβ measures. The additional effects of possible confounding variables (age, sex, presence of ApoE4, and family history of dementia) were considered using forward selection. Because of the skewed distributions of the Aβ measures, Aβ₄₂, Aβ₄₀, and Aβ₄₂/Aβ₄₀ were included in models in a number of different ways to explore the effect on the results. Each Aβ measure was included as follows: as a continuous variable on the logarithmic scale, as a binary category variable defined by the median, and as a continuous variable defined by the quartiles of the distribution (1,2,3,4). Quartiles were also included as categorical variables to enable estimation of hazard ratios relative to the highest quartiles and to aid in exploring whether a linear association was reasonable. Sensitivity of results to removal of the outlying Aβ measures (upper and lower 2%) was also considered in the continuous variable models. No adjustments were made for multiple testing in these exploratory analyses.

Additional regression models were used to explore the relationship between Aβ measures and change in cognition over a subsequent period of approximately five years in a subset of 379 subjects who were administered the DRS within six months of a plasma Aβ measurement. The total DRS raw score was converted to an age-adjusted scaled score based on data from Mayo's Older American Normative Scores (MOANS; Ivnik et al., J Clin Exp Neuropsychol, 19(5):676-91 (1997)). Because follow-up intervals varied, annualized rates of cognitive change were used as the outcome variable in least-squares regression analyses that were conducted in a manner similar to the time-to-conversion analyses.

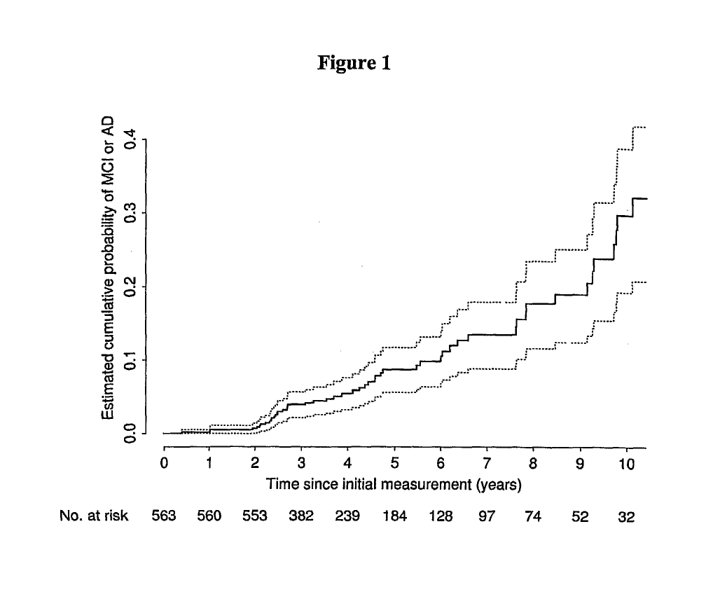

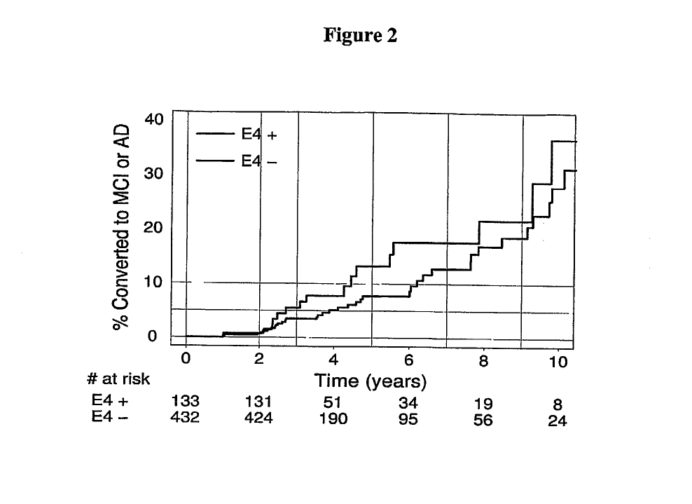

Fifty-three of the 563 subjects were observed to develop MCI or AD. The estimated cumulative incidence in this cohort of subjects (FIG. 1) with median age 78 at start of follow-up was 5% at four years, with a 95% confidence interval (CI) ranging from 3% to 8%. It reached 11% (95% CI: 7% to 14%) at six years, was 18% (95% CI: 12 to 24%) at 8 years, and was 30% (95% CI: 19% to 39%) at 10 years of follow-up. Both advancing age and presence of the ApoE4 allele revealed evidence of association with incidence of MCI or AD (Table 2), whereas family history did not. The relationship between the presence or absence of the ApoE4 allele and the cumulative incidence of MCI or AD was determined and presented graphically (FIG. 2).

TABLE 2
Associations of time to MCI or AD with age,
sex, presence of ApoE4, and family history.

| | Single variable model | | | Adjusting for age and ApoE4 | | |
|---|---|---|---|---|---|---|
| | Relative | | | Relative | | |
| Variable | Risk | 95% CI | P-value | Risk | 95% CI | P-value |
| Age (10 years) | 4.10 | 2.58-6.52 | <0.001 | 4.27 | 2.66-6.85 | <0.001 |
| ApoE4 | 1.80 | 1.02-3.16 | 0.041 | 1.93 | 1.09-3.39 | 0.023 |
| Family history | 1.43 | 0.83-2.46 | 0.20 | 1.13 | 0.65-1.99 | 0.67 |
| Sex (Female) | 1.35 | 0.73-2.49 | 0.34 | 0.80 | 0.66-2.39 | 0.49 |

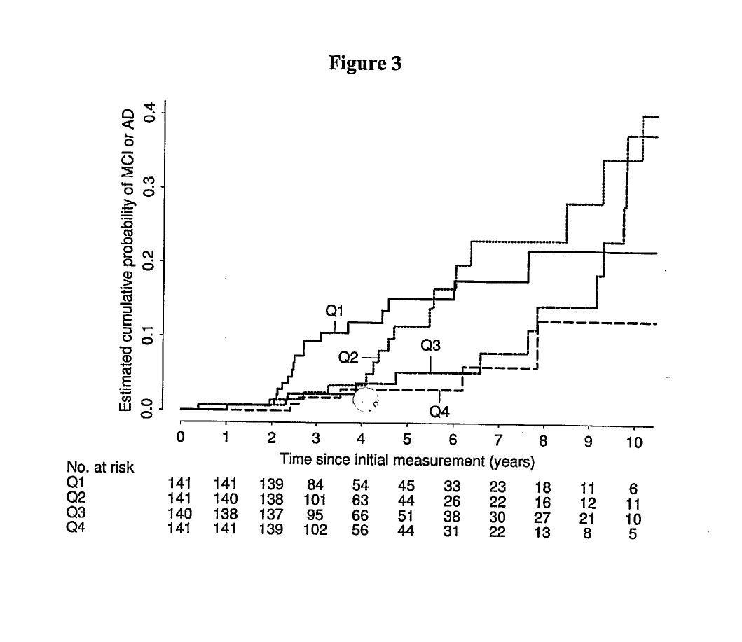

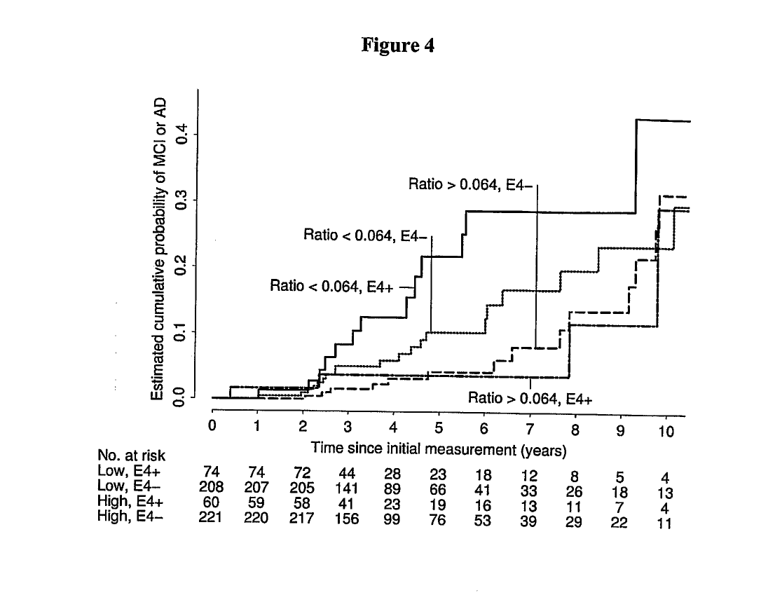

The plasma Aβ₄₂/Aβ₄₀ ratio exhibited an association with conversion to MCI or AD (Table 3); however, neither Aβ₄₂ nor Aβ₄₀ exhibited any such association. The risk of MCI or AD for subjects with an Aβ₄₂/Aβ₄₀ ratio in the lowest quartile was estimated to be three times the risk for subjects with a ratio in the highest quartile (p=0.010), and this association persisted (p=0.038) after adjusting for age and ApoE4 (Table 3). The striking relationship between the Aβ₄₂/Aβ₄₀ ratio and cumulative incidence of MCI or AD was presented graphically (FIG. 3). Subjects whose Aβ₄₂/Aβ₄₀ ratio was in the lowest quartile (Q1: <0.05) reached a 10% cumulative incidence by three years, while those with an Aβ₄₂/Aβ₄₀ ratio in Q2 did not reach this incidence for approximately five years, and those with an Aβ₄₂/Aβ₄₀ ratio in Q3 and Q4 did not reach this incidence for approximately eight years. This pattern suggested a deviation from the proportional hazards assumption of the models, but formal significance tests indicated no significant deviation. Elderly subjects with an ApoE4 allele are at increased risk for AD, but approximately 50% of those with an ApoE4 allele do not develop AD. As demonstrated herein, the Aβ₄₂/Aβ₄₀ ratio was highly effective in separating the ApoE4 carriers who developed MCI or AD from those who did not. After five years of follow-up, over 20% of subjects with an ApoE4 allele and a low (below median) Aβ₄₂/Aβ₄₀ ratio had developed AD (FIG. 4). In contrast, only 3% of the ApoE4 carriers with a high (above median) Aβ₄₂/Aβ₄₀ ratio developed AD by five years. Remarkably, more subjects without an ApoE4 allele who had a low Aβ₄₂/Aβ₄₀ ratio had developed AD by five years (10%) than subjects with an ApoE4 allele who had a high Aβ₄₂/Aβ₄₀ ratio (3%).

TABLE 3
Associations of time to MCI or AD with Aβ₄₂/Aβ₄₀.

| | Single variable model | | | Adjusting for age and apoE4 | | |
|---|---|---|---|---|---|---|
| | Relative | | | Relative | | |
| Variable | Risk | 95% CI | P-value | Risk | 95% CI | P-value |
| Ratio (<0.064) | 1.80 | 1.03-3.13 | 0.038 | 1.48 | 0.85-2.60 | 0.17 |
| Ratio* | | | | | | |
| Q1 | 3.33 | 1.23-9.04 | | 3.04 | 1.12-8.30 | |
| Q2 | 2.97 | 1.09-8.10 | 0.010 | 2.43 | 0.89-6.63 | 0.038 |
| Q3 | 2.09 | 0.75-5.84 | | 2.33 | 0.83-6.54 | |
| Q4 | 1.00 | — | | 1.00 | — | |
| Log Ratio (Halving) | 1.43 | 1.00-2.04 | 0.049 | 1.39 | 0.95-2.03 | 0.084 |
| Log Ratio (Halving) | 1.55 | 0.92-2.61 | 0.10 | 1.46 | 0.82-2.59 | 0.20 |
| extremes removed \hat{ } | | | | | | |

*Upper quartile, Q4, is reference. P-value from model with quartiles as a continuous variable.
\hat{ }Extremes were defined as the upper and lower 2% of Aβ42/Aβ40 measures

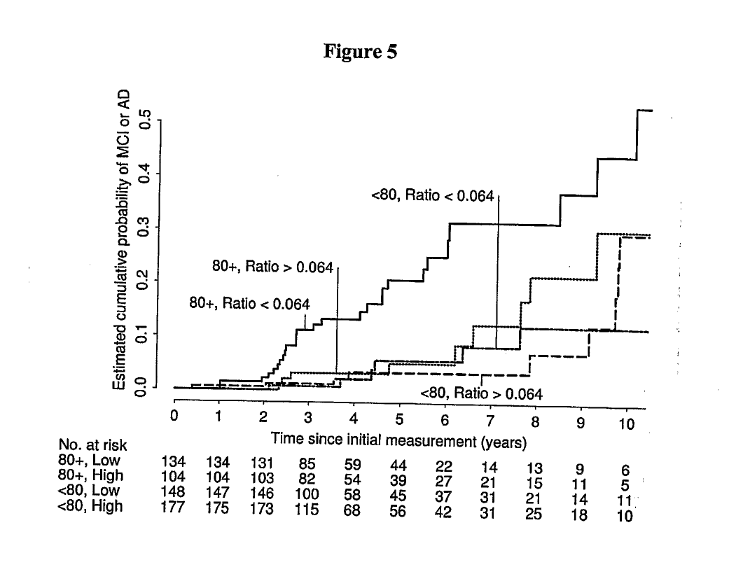

Combining age and Aβ₄₂/Aβ₄₀ ratio was also highly effective in separating subjects who developed MCI or AD from those who did not. As demonstrated herein, 20% of older subjects (age ≧80 years) with a low (below median) Aβ₄₂/Aβ₄₀ ratio developed AD or MCI by five years (FIG. 5). In contrast, less than 5% of all other subjects developed AD within five years.

The regression analysis using change in DRS score as the outcome variable in a subset of subjects resulted in confirmatory findings. Adjusting for age and years of education, the Aβ₄₂/Aβ₄₀ ratio exhibited an association with a decline in cognition, with lower ratios being associated with greater declines (Table 4). For example, subjects with ratios lower than the median tended to have memory declines that were on average 0.20 lower per year than those with ratios higher than the median.

TABLE 4
Associations of Aβ₄₂/Aβ₄₀ with change in DRS total MOANS score.

| | | | | Adjusting for age and educatior | | |
|---|---|---|---|---|---|---|
| | Single variable model | | | level | | |
| Variable | Beta^{†} | 95% CI | P-value | Beta | 95% CI | P-value |
| Ratio (<median) | −0.22 | −0.36-−0.09 | <0.001 | −0.20 | −0.33-−0.07 | 0.003 |
| Quartiles* | | | | | | |
| Q1 | −0.19 | −0.38-0.00 | | −0.15 | −0.34-0.04 | |
| Q2 | −0.18 | −0.36-0.01 | 0.007 | −0.12 | −0.31-0.07 | 0.021 |
| Q3 | −0.08 | −0.10-0.27 | | 0.12 | −0.07-0.30 | |
| Q4 | 0.00 | — | | 0.00 | — | |
| Log Ratio | −0.10 | −0.19-−0.10 | 0.037 | −0.08 | −0.17-0.01 | 0.076 |
| (Halving) | | | | | | |
| Log Ratio | −0.14 | −0.27-−0.01 | 0.032 | −0.12 | −0.24-−0.01 | 0.077 |
| (Halving) extremes | | | | | | |
| removed \hat{ } | | | | | | |

*Upper quartile, Q4, is reference. P-value from model with quartiles as a continuous variable
^{†}Estimated coefficient of annualized change in DRS score. Negative coefficients indicate greater declines in participants with lower Aβ₄₂/Aβ₄₀ measures
\hat{ }Extremes were defined as the upper and lower 2% of Aβ₄₂/Aβ₄₀ measures

These results demonstrate that, after correcting for age and ApoE4, subjects with plasma Aβ₄₂/Aβ₄₀ ratios in the lowest quartile as compared to the highest quartile had an estimated relative risk of 3.0 (p=0.038). After adjusting for age and years of education, participants with Aβ₄₂/Aβ₄₀ ratios lower than the median had greater cognitive decline (by 0.20 points per year) than those with ratios higher than the median (p=0.003). In participants over age 80, the five-year cumulative incidence of AD or MCI was approximately 20% for those with Aβ₄₂/Aβ₄₀ ratios in the lower half compared to approximately 5% for those with ratios in the upper half.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for determining whether or not a mammal is susceptible to develop mild cognitive impairment or Alzheimer's disease, said method comprising determining whether or not said mammal contains plasma with an Aβ₄₂ to Aβ₄₀ ratio that is less than 0.082, wherein the presence of said ratio indicates that said mammal is susceptible to develop mild cognitive impairment or Alzheimer's disease.

2. The method of claim 1, wherein said mammal is a human.

3. The method of claim 1, wherein said mammal is a human not diagnosed with mild cognitive impairment or Alzheimer's disease.

4. The method of claim 1, wherein said method comprises determining whether or not said mammal is susceptible to develop mild cognitive impairment.

5. The method of claim 1, wherein said method comprises determining whether or not said mammal is susceptible to develop Alzheimer's disease.

6. The method of claim 1, wherein said method comprises determining whether or not said mammal contains plasma with an Aβ₄₂ to Aβ₄₀ ratio that is less than 0.064.

7. The method of claim 1, wherein said method comprises determining whether or not said mammal contains plasma with an Aβ₄₂ to Aβ₄₀ ratio that is less than 0.05.

8. The method of claim 1, wherein said determining step comprises using an enzyme-linked immunosorbant assay.

9. The method of claim 1, wherein said method comprises classifying said mammal as being susceptible to develop mild cognitive impairment or Alzheimer's disease within two years.

10. The method of claim 1, wherein said method comprises classifying said mammal as being susceptible to develop mild cognitive impairment or Alzheimer's disease within two years when said Aβ₄₂ to Aβ₄₀ ratio is less than 0.05.

11. The method of claim 1, wherein said method comprises classifying said mammal as being susceptible to develop mild cognitive impairment or Alzheimer's disease within four years.

12. The method of claim 1, wherein said method comprises classifying said mammal as being susceptible to develop mild cognitive impairment or Alzheimer's disease within four years when said Aβ₄₂ to Aβ₄₀ ratio is between 0.05 and 0.064.

13. The method of claim 1, wherein said method comprises classifying said mammal as being susceptible to develop mild cognitive impairment or Alzheimer's disease within ten years.

14. The method of claim 1, wherein said method comprises classifying said mammal as being susceptible to develop mild cognitive impairment or Alzheimer's disease within ten years when said Aβ₄₂ to Aβ₄₀ ratio is between 0.064 and 0.082.

15. The method of claim 1, wherein said method comprises classifying said mammal as not being susceptible to develop mild cognitive impairment or Alzheimer's disease within ten years when said Aβ₄₂ to Aβ₄₀ ratio is greater than 0.082.

16. The method of claim 1, wherein said method comprises, before said determining step, obtaining a plasma sample from said mammal, wherein said determining step comprises determining whether or not said plasma sample contains said Aβ₄₂ to Aβ₄₀ ratio that is less than 0.082.
